(19) United States
(12) Patent Application Publication
(10) Pub. No.: US 2015/0133870 A1
(43) Pub. Date: May 14, 2015

(54) SAFETY SYRINGE

(71) Applicant: Ansell Limited, Richmond (AU)

(72) Inventors: Jamie Ashworth, Freehold, NJ (US); Joel Melvyn Goldenberg, Upper Saddle River, NJ (US); David Meck, Sewell, NJ (US); Rahul Eapen, West Palm Beach, FL (US); Wendell Franke, Gallatin, TN (US)

(21) Appl. No.: 14/540,441

(22) Filed: Nov. 13, 2014

Related U.S. Application Data
(60) Provisional application No. 61/904,221, filed on Nov. 14, 2013.

(51) Int. Cl. A61M 5/32; A61M 5/50; A61M 5/31

(52) U.S. Cl. CPC A61M 5/3245; A61M 5/3135; A61M 5/3269; A61M 5/3271; A61M 5/50; A61M 2005/3247
USPC 604/197

(57) ABSTRACT

Safety syringes are disclosed, wherein a needle, at least one locking track and at least one tab disposed on an exterior surface of the barrel, wherein the barrel is in fluid communication with the needle, a plunger capable of fitting into the barrel, and a hollow sleeve having a first opening on a first end and a second opening on an opposite end, and having at least one protrusion on an interior surface of the hollow sleeve, wherein the tab on the at least one locking track on the barrel is adapted to engage the at least one protrusion of the hollow sleeve, thereby locking a position of the barrel with respect to a position of the hollow sleeve.

### CROSS-REFERENCES TO RELATED APPLICATIONS

This application claims the benefit of U.S. Provisional Application No. 61/904,221, filed Nov. 14, 2013, which is incorporated by reference in its entirety.

### BACKGROUND

1. Field

Embodiments of the present invention generally relate to a disposable safety syringe apparatus and, more particularly, to a disposable safety syringe having a sleeve comprising at least one locking mechanism.

2. Description of the Related Art

The prevalence of syringes in the medical field makes needle stick injuries a frequent concern for medical professionals. In certain situations, such as in a surgical environment, many injections, from separate syringes, are given to patients, increasing the number of exposed syringes in the environment. Accidental needle sticks, from needles left uncapped and exposed for repetitive use in a procedure, increasing the risk of inadvertently penetrating the glove or skin of the surgeon, surgeon's assistant, or other medical personnel, are not uncommon. Furthermore, uncapped needles can compromise the receptacles used to dispose of used syringes and needles, such as bags, leading to injuries.

A syringe apparatus that enables temporary locking features for locking during intervals between repetitive uses and permanent locking features for use when the apparatus is ready to be discarded are proposed as advances in the art.

### SUMMARY OF EMBODIMENTS OF THE INVENTION

Embodiments of the invention comprise safety syringes having a needle, at least one locking track on a barrel and at least one tab disposed on an exterior surface of the barrel, wherein the barrel is in fluid communication with the needle, a plunger capable of fitting into the barrel, and a hollow sleeve having a first opening on a first end and a second opening on an opposite end, and having at least one protrusion on an interior surface of the hollow sleeve, wherein the tab on the at least one locking track on the barrel is adapted to engage the at least one protrusion of the hollow sleeve, thereby locking a position of the barrel with respect to a position of the hollow sleeve, are disclosed. Methods for using safety syringes are disclosed.

Various advantages, aspects, and novel features of the present disclosure, as well as details of exemplary embodiments thereof, will be understood more fully from the following description and drawings.

### BRIEF DESCRIPTION OF THE DRAWINGS

FIG. 1 depicts a perspective view of an assembled safety syringe apparatus according to one or more embodiments of the invention;

FIG. 2 depicts a perspective view of the sleeve of FIG. 1;

FIG. 2A depicts a cross-sectional view of the sleeve of FIG. 2 taken along line 2A-2A, according to embodiments of the invention;

FIG. 3 depicts a perspective view of the barrel of FIG. 1, according to one or more embodiments of the invention;

FIG. 4A depicts a perspective view of the safety syringe apparatus of FIG. 1 depicting the sleeve in a first position, according to embodiments of the invention;

FIG. 4B depicts a perspective view of the safety syringe apparatus of FIG. 1 depicting the sleeve and a temporary lock in a first safe position, according to embodiments of the invention;

FIG. 5 depicts a perspective view of the safety syringe apparatus of FIG. 1 depicting the sleeve and a permanent lock in a second safe position, according to embodiments of the invention;

FIG. 6 depicts a perspective view of the safety syringe of FIG. 1 depicting a temporary locking feature in an operating position, according to one or more embodiments of the invention;

FIG. 7 depicts a flow diagram of a method for using the safety syringe apparatus according to embodiments of the invention;

FIG. 8 depicts a perspective view of an exemplary assembled safety syringe apparatus having a sleeve in an operating position, according to embodiments of the invention;

FIG. 9 depicts a perspective view of the sleeve and the barrel of FIG. 8, between an operating position and a safe position; according to embodiments of the invention;

FIG. 10 depicts a perspective view of the sleeve and the barrel of FIG. 8 in a temporarily locked safe position, according to embodiments of the invention;

FIG. 11 depicts a perspective view of the sleeve and the barrel of FIG. 8 in a permanently locked safe position, according to embodiments of the invention;

FIG. 12 depicts a perspective view of a collar and a barrel assembly, according to embodiments of the invention;

FIG. 13 depicts an exploded perspective view of a two-piece sleeve for mating with a barrel, according to embodiments of the invention; and

FIG. 14 depicts an exploded perspective view of a two-piece sleeve for mating with the barrel in accordance with embodiments of the invention.

While the method and/or apparatus described herein are exemplary embodiments, those skilled in the art will recognize that the method and apparatus for safety syringes are not limited to the embodiments or drawings described. Alternately, modifications, equivalents and alternatives falling within the spirit and scope of the method and apparatus are defined by the appended claims. Headings used herein are for organizational purposes only and do not limit the scope of the description or the claims.

The word “may” is used in a permissive sense (i.e., meaning having the potential to), rather than the mandatory sense (i.e., meaning must), and the words “include,” “including,” and “includes” mean including, but not limited to. Furthermore, throughout the drawings, reference numerals used to refer to like parts, components, and structures may be common to one or more figures.

### DETAILED DESCRIPTION

Embodiments according to the present invention generally comprise a syringe capable of being permanently locked or temporarily locked in a use position or a non-use position. For example, a syringe comprises a hollow sleeve joined therewith, which further comprises at least one protrusion capable of engaging at least one notched track on a barrel of the syringe. When the syringe is capable of being used for procedures, i.e., for drawing fluids into the syringe (from a patient or container) and/or injecting fluids from the syringe into a patient or a container, by manipulating a plunger joined to the barrel, a needle of the syringe protrudes at least partially out of the hollow sleeve. When the syringe is not being used and is in a safe condition, i.e., the needle cannot accidentally stick a person, the needle is completely withdrawn into the hollow sleeve. The barrel and the hollow sleeve comprise features permitting the syringe to be locked in a temporary lock when the syringe is capable of being used as well as features permitting the syringe to be locked in a temporary lock and/or a permanent lock when the syringe is in a safe condition. Once a syringe is in a permanently locked safe condition, the syringe cannot be used, i.e., the needle cannot be forced from the hollow sleeve without destroying the syringe, rendering it useless.

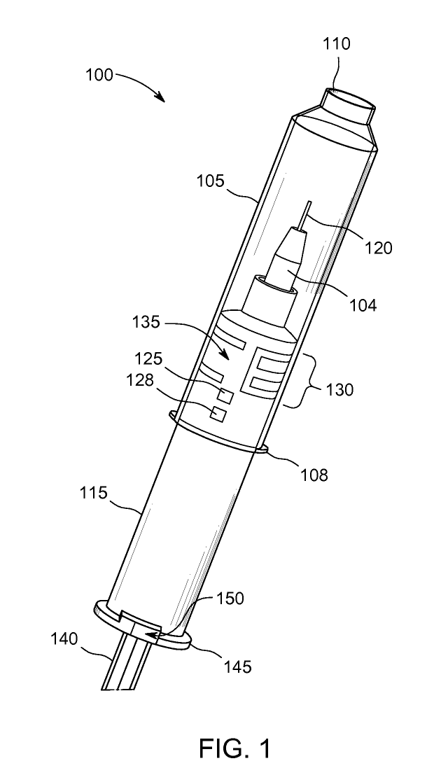

FIG. 1 depicts a perspective view of an assembled safety syringe apparatus 100 according to one or more embodiments of the invention. The safety syringe apparatus 100 comprises a barrel 115 inserted into a sleeve 105. The sleeve 105 is hollow and further defines a first opening 110 on a first end of the sleeve 105. On an interior surface of the sleeve 105 are a first protrusion 125 and a second protrusion 128. As shown, the interior surface of the sleeve has the first protrusion 125, and the second protrusion 128. In practice, safety syringe apparatus 100 will operate wherein the sleeve 105 comprises only the first protrusion 125 or the second protrusion 128. The barrel 115 comprises a first end 145 into which a syringe plunger 140 is inserted and a second end comprising a needle seat 104, which may be tapered and a needle 120. The needle 120 defines a fluid channel (not shown) that is held in the barrel 115, which is capable of drawing and injecting fluids.

The barrel 115 exterior further comprises at least two tracks. The tracks 130 are formed on the exterior of the barrel 115 and slidably fit with the first protrusion 125 and the second protrusion 128 located on the interior of the sleeve 105. When the needle 120 is not in use, the protrusions 125, 128 can engage the tracks 130 and the needle 120 becomes fully enclosed within the sleeve 105.

The tracks 130 also include a path 135 for the protrusions to pass through such that the protrusions 125, 128 pass through the path 135 and the needle 120 extends through the sleeve 105 at the first opening 110 when the needle is in use. In alternative embodiments, the tracks 130 may be located on the sleeve 105 and the protrusions 125, 128 on the exterior of the barrel 115 (not shown).

The sleeve 105 is slightly larger in inner diameter than the outer diameter of the barrel 115, allowing a tight fit and such that the first protrusion 125 and second protrusion 128 may engage the tracks 130 on the inner surface of the sleeve when the barrel 115 is inserted into the sleeve 105. As discussed further below, in a first position (i.e., when the needle is capable of being used for injecting or taking up a fluid) the barrel 115 is positioned within the sleeve 105, i.e., the needle 120 extends/protrudes from the first opening 110 of the sleeve 105. The barrel 115 is releasably secured to the sleeve 105 through an interlocking rib 108 located at the distal end of the first opening 110 such that the rib 108 fits into a slot 150 on the distal end of the barrel 115 to prevent movement of the barrel 125 within the sleeve 105 during use. In a second position (i.e., the needle is retracted and disengaged and therefore not in use), when the protrusions 125, 128 are engaged with the tracks 130, the needle 120 is fully enclosed by the sleeve 105 (i.e., does not protrude from the first opening 110).

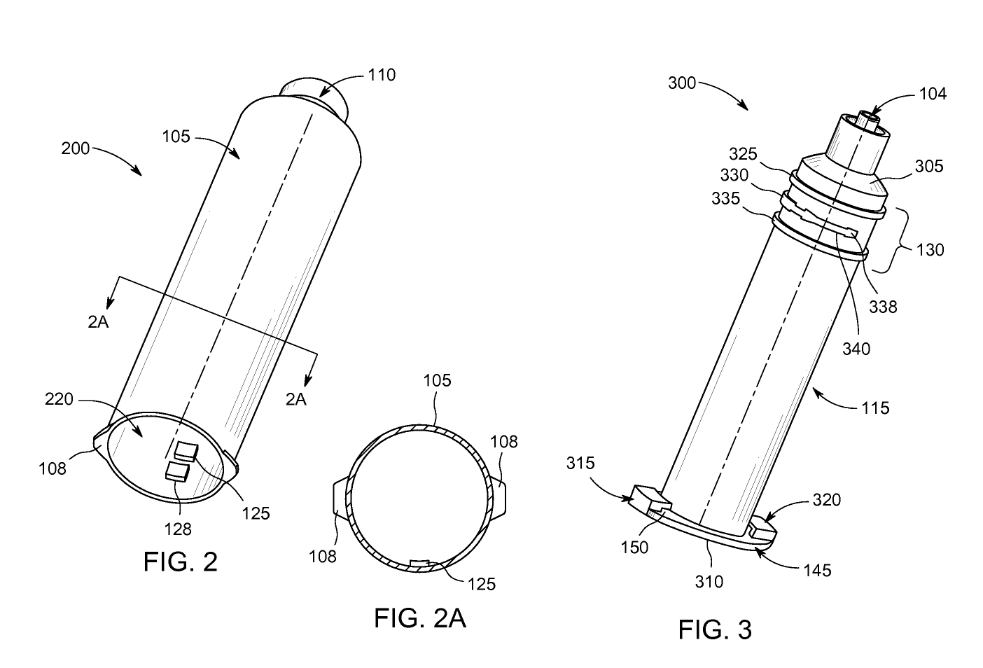

FIG. 2 depicts a perspective view 200 of the sleeve 105 of FIG. 1. The sleeve 105 comprises a first opening 110 and a second opening 220 wherein the second opening 220 is larger than the first opening 110. The second opening 220 has an inner diameter large enough to allow the barrel 115 of FIG. 1 to be inserted. The sleeve 105 is substantially cylindrical and capable of receiving the barrel 115. The exterior of the sleeve 105 at the second opening 220 also comprises at least one rib 108 that extends away from sleeve 105. The first protrusion 125 and the second protrusion 128 are formed on the interior of the sleeve 105. The first protrusion 125 and the second protrusion 128 in this exemplary embodiment are square but may alternatively be other shapes (circular, triangular, and the like) or have rounded corners. Protrusions comprising any shape may be tapered.

FIG. 2A depicts a cross-sectional view of the sleeve of FIG. 2 taken along line 2A-2A, according to embodiments of the invention. The sleeve 105 is shown with two ribs 108 disposed on an outer surface of the sleeve 105 and at least one first protrusion 125 disposed on an internal surface of the sleeve 105. The sleeve 105 may comprise at least one protrusion or more than one protrusion on the internal surface.

FIG. 3 depicts a perspective view 300 of the barrel 115 of FIG. 1, according to one or more embodiments of the invention. The barrel 115 comprises a needle seat 104 and a tapered barrel portion 305 on the distal end of the barrel 115, which further comprises an opening 310 for receiving a syringe plunger 140 (discussed above). Adjacent to the syringe plunger opening 310 are at least grips 315, 320, for grasping by a user's fingers. The grips 315, 320 also comprise a slot 150 for locking with the rib 108 of FIG. 1 when the syringe is in the first position. The barrel 115 comprises tracks 130, disposed between the tapered barrel portion 305 and the grips 315, 320. Embodiments according to the invention include wherein the barrel 115 comprises tracks 130 having at least two guiding tracks 325, 335 between which a locking track 330 is positioned. The guiding tracks 325, 335 are about the same length and do not traverse an entire circumference of the barrel 115. The locking track 330 traverses less of the circumference of the barrel 115 than does the guiding tracks 325, 335 and further comprises at least one tab or notch having a wide tapered portion 338 followed by a thinned area comprising at least one temporary locking tab or notch 340. Two locking tabs or notches 340 are shown. The distance between the guiding tracks 325, 335 and the locking track 330 allow the first protrusion 125 and the second protrusion 128 of the sleeve 105 (discussed above) to slidably fit therein.

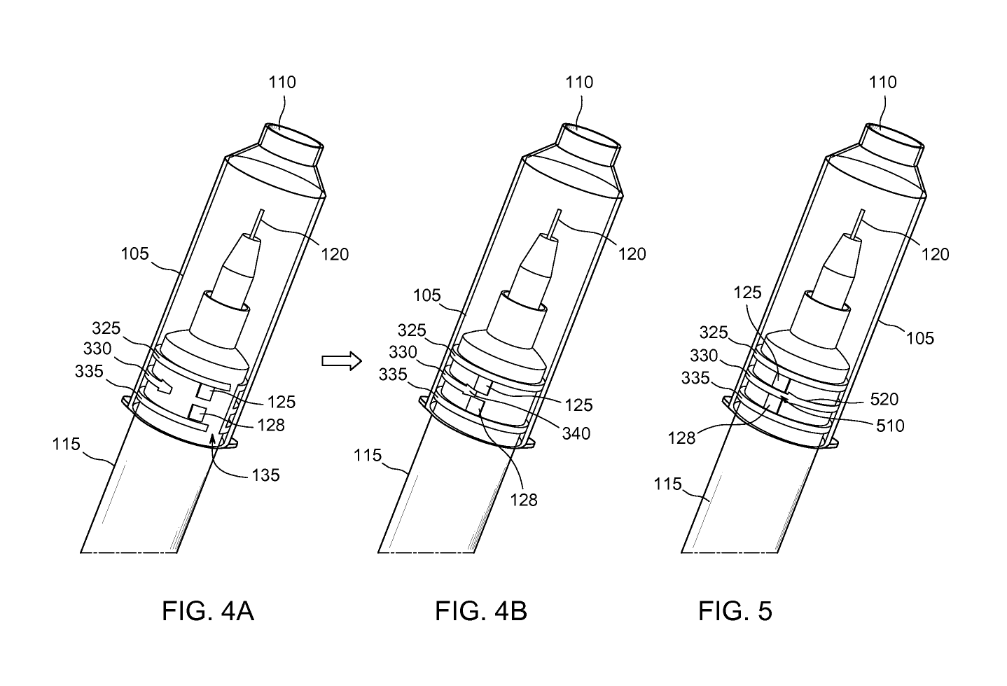

FIGS. 4A and 4B are perspective views of the safety syringe apparatus 100 of FIG. 1 depicting a temporary lock in a second position, according to embodiments of the invention. FIG. 4A depicts a perspective view of the safety syringe apparatus of FIG. 1, depicting the sleeve 105 in a first position, according to embodiments of the invention. FIG. 4A illustrates the protrusions 125, 128 engaging the locking track 330 after entering via the path 135. The barrel 115 slides linearly with respect to the sleeve 105 and may be rotated when the protrusions 125, 128 are adjacent the guiding tracks 325, 335. By following the guiding tracks 325, 335, the protrusions 125, 128 reach the locking track 330 in FIG. 4B located between the guiding tracks 325, 335.

FIG. 4B depicts a perspective view of the safety syringe apparatus of FIG. 1 depicting the sleeve and a temporary lock in a first safe position, according to embodiments of the invention. FIG. 4B illustrates the temporary locking feature when the needle 120 is not in use, i.e., the second position. The temporary lock is engaged where protrusions 125, 128 slide on the locking track 330 to tab or notch 340. The tab or notch 340 is shallow enough such that the protrusions 125, 128 may rotate in the reverse direction with additional twisting force to exit the temporary locking tab or notch 340 and towards the path 135 to use the needle 120, whereupon the sleeve 105 can advance or retract linearly, i.e., return to a position for further use. Also, syringes having temporary locks, unlike syringes having no lock, cannot accidentally stick a person if used correctly, according to embodiments of the invention.

FIG. 5 depicts a perspective view of the safety syringe apparatus of FIG. 1 depicting the sleeve and a permanent lock in a second safe position, according to embodiments of the invention. FIG. 5 depicts the protrusions 125, 128 reaching the permanent locking tab or notch 510 located on the locking track 330. The permanent locking tab or notch 510 is comprised of thinned areas of the locking track 330 such that the protrusions 125, 128 cannot slide back to the path 135 to use the needle 120 once rotated past the tab or notch 510. The permanent locking notch 510 comprises a wider locking track portion 520 followed by a thinned locking track area comprising the permanent locking tab or notch 510 with wider walls, thereby immobilizing protrusions 125, 128. As the protrusions 125, 128 slide (as the sleeve 105 is rotated) on the guiding tracks 325, 335, the protrusions 125, 128 eventually encounter the wider locking track 520 that is tapered such that the protrusions 125, 128 fall into the permanent locking notches 510 along the locking track 330. Specifically, as the barrel 115 is rotated with respect to the sleeve 105, the protrusions 125, 128, slide along the locking track 330, which is tapered and gets wider at the wider locking track portion 520, which the protrusions 125, 128 can be forced past. However, it is not possible to rotate the barrel 115 in the reverse direction because the wider locking track portion 520 is too wide to allow the protrusions 125, 128 to be forced past the wider locking track portion 520. Therefore, the barrel 115 cannot be advanced or retracted linearly nor rotated. Therefore, the needle 120 is thus permanently immobilized and enclosed by the sleeve 105 and can no longer be used and can never protrude from the sleeve 105 without destroying the sleeve 105 and/or barrel 115, rendering the safety syringe 100 useless. Therefore, stolen syringes having permanent locks, unlike other stolen syringes that can be used leading to infections, cannot infect a person.

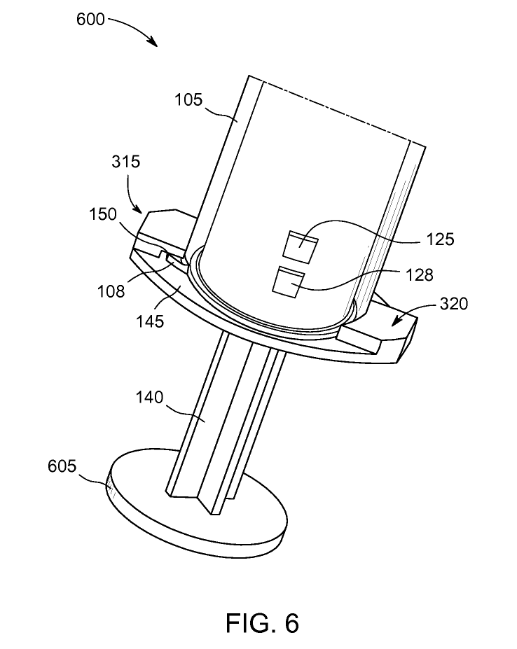

FIG. 6 depicts a perspective view of the safety syringe apparatus of FIG. 1, depicting a temporary locking feature in an operating position, according to embodiments of the invention. In the first position (when the needle 120 is capable of being in use), the barrel 115 may be releasably secured to the sleeve 105 via the rib 108 and the slot 150. The coupling of the rib 108 and the slot 150 allows the barrel 115 and the sleeve 105 to operate as a single unit, i.e., neither moves, when the syringe plunger 140 moves due to forces applied to a syringe plunger end 605, for example, during injection of fluids. The barrel 115 is secured by slide lock fit via at least one rib 108 on the sleeve 105 and the slot 150 formed in the grips 315, 320 on the barrel 115. Alternative embodiments of the grips 315, 320 may secure the sleeve 105 to the barrel 115. As shown, there is no temporary lock comprising a locking track on a barrel to be engaged by the protrusions 125, 128, although such an embodiment is possible, as discussed below.

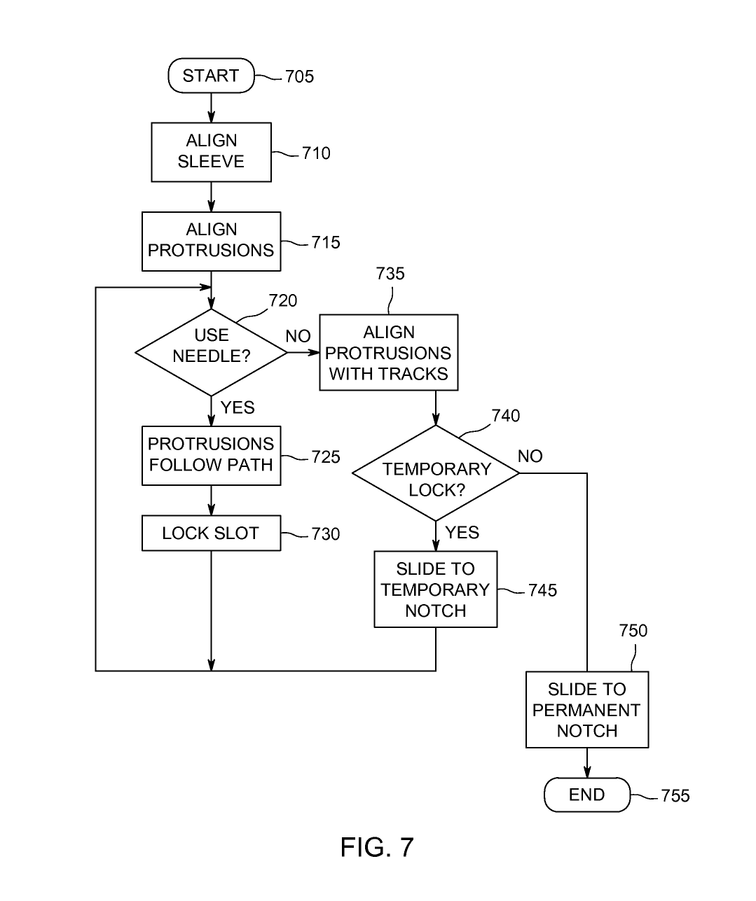

FIG. 7 depicts a flow diagram of a method 700 for using the safety syringe apparatus 100 according to one or more embodiments of the invention. In some embodiments, the barrel 115 and the sleeve 105 may be pre-assembled as the safety syringe apparatus 100. FIG. 7 discloses an embodiment to assemble the barrel 115 and sleeve 105 as well as operation of the resulting safety syringe apparatus 100. The method 700 begins at step 705 and continues to step 710 wherein the needle 120, incorporated within the barrel 115, are aligned and disposed within the sleeve 105. At step 715, the protrusions 125, 128 disposed on the interior of the sleeve 105, as discussed above, are aligned with the path 135.

The method 700 continues to step 720 wherein a decision is made whether the needle is to be used (i.e. engaged, injected, administered and the like), if the answer is yes, the method continues to step 725 wherein the protrusions pass through the path 135 until the slot 150 is able to lock with the rib 108. At step 730, the slot 150 and rib 108 are slidably locked together such that the barrel 115 and sleeve 105 can operate as a single unit with the syringe plunger 140. The method 700 continues to step 720 to determine whether the needle is to be engaged/used.

Should the method 700 determine the needle 120 is not being used at step 720, the method continues to step 735 wherein the protrusions 125, 128 are aligned with the tracks 130. The method 700 then determines whether to lock the safety syringe temporarily at step 740. If the answer is yes, the method 700 continues to step 745 and the protrusions 125, 128 are slid to the temporary locking notch 340 and remains in this position until a decision to use the needle 120 at step 720 is made. If however, the method 700 decides the locking mechanism is not temporary at step 740, for example, after the syringe apparatus 100 has been used and will be discarded, the method 700 continues to step 750 to slide into the permanent locking notch 510 and the method 700 ends at step 755.

Embodiments according to the invention comprise three locking positions. The first position is an operating position, in which the sleeve 105 is drawn back, exposing the needle 120 (so that the safety syringe apparatus 100, or other safety syringe apparatus described herein can be used to inject fluids, for example). The barrel 415 and the sleeve 105 may be locked temporarily. The second position is a safe, temporarily locked position, for which the sleeve 105 is advanced, i.e., wherein the needle 120 is obscured because the sleeve 105 is advanced forward, preventing accidental sticks. The third position is a safe position and comprises wherein the sleeve 105 is advanced forward so that the needle 120 is obscured, however, the locking is permanent, indicating that the safety syringe 100 cannot be unlocked without destroying the safety syringe 100.

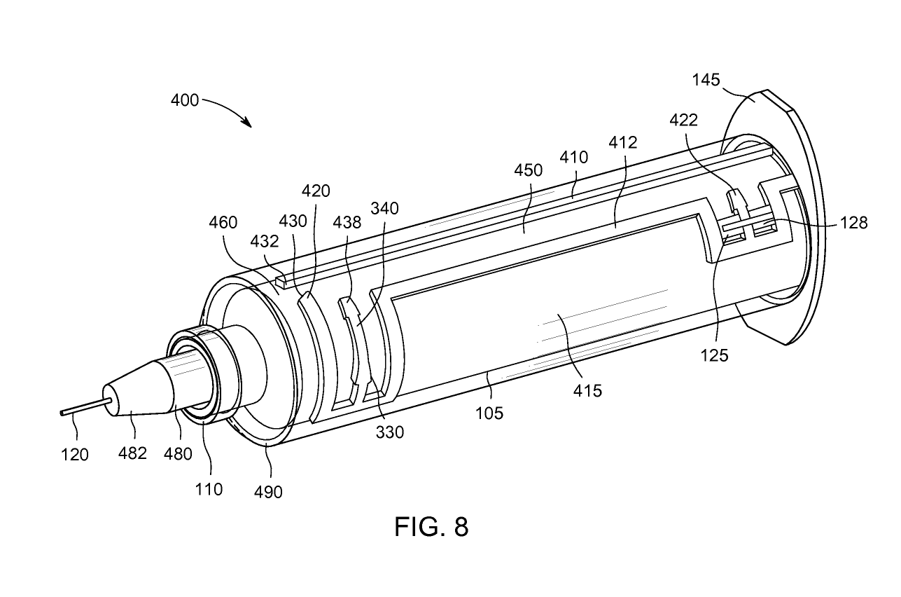

FIG. 8 depicts a perspective view of an exemplary assembled safety syringe apparatus 400 having a sleeve in an operating position, according to embodiments of the invention. Similar to embodiments discussed above, the safety syringe apparatus 400 comprises a barrel 415 inserted into the sleeve 105, which, as shown is made of a transparent or translucent material, such as polyethylene, polypropylene, polycarbonate, acrylics, or cellulosic-based materials. Other materials, such as copolymers, are contemplated herein, such as styrene-acrylonitrile (SAN) and acrylonitrile-butadiene-styrene (ABS) polymeric materials. The barrel 415 may comprise these same polymeric materials. The sleeve 105 comprises a first opening 110 on a distal end 490 of the sleeve 105, which allows a needle 120 to pass therethrough, similar to embodiments of the invention discussed above. The interior of the sleeve 105 comprises a first protrusion 125 and a second protrusion 128. The needle 120 defines the fluid channel in the barrel 415 for injecting and/or drawing fluids.

The barrel 415 exterior further comprises at least two tracks. For example, the barrel 115 comprises guide tracks 410 and 412. The first protrusion 125 and the second protrusion 128, located on the interior of the sleeve 105, can be engaged slidably through a space 460 of the barrel 415. In some embodiments according to the present invention, the ends 430 and 432 of the guide tracks 410 and 412 are tapered, so that space 460 is larger, and therefore the first protrusion 125 and second protrusion 128 can more easily slide into the space 460.

The sleeve 105 is slightly larger in inner diameter than the diameter of the barrel 415 such that the first protrusion 125 and the second protrusion 128, disposed on the sleeve 105, may engage the guide tracks 410, 412 as the barrel 415 is inserted into the sleeve 105. As will be further discussed below, in a first position, for example, the needle is capable of injecting or charging with a fluid, the barrel 415 is positioned within the sleeve 105 such that the needle 120 extends/protrudes from the first opening 110 of the sleeve 105. The barrel 415 is secured to the sleeve 105 through the interaction between the temporary locking notch 422 (disposed on a locking track) and the first protrusion 125 and the second protrusion 128. As illustrated, the barrel 415 and the sleeve 105 are temporarily locked, preventing movement of the barrel 415 within the sleeve 105 during injection or charging. Embodiments according to the invention also comprise wherein the needle 120 has an extended end 480 adjacent to a tapered tip 482. Because the extended end 480 is substantially cylindrical and has a length of approximately 0.25-1.0 inches, it can be gripped by a user, which allows it to be easily screwed into, joined, or otherwise connected with luer locks, which are typically disposed within a sleeve, as is known to those in the art.

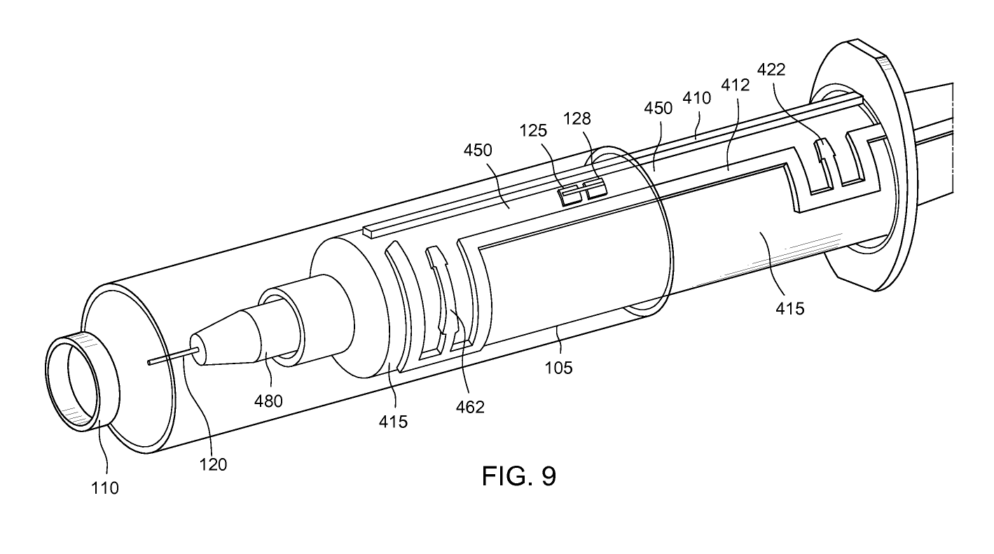

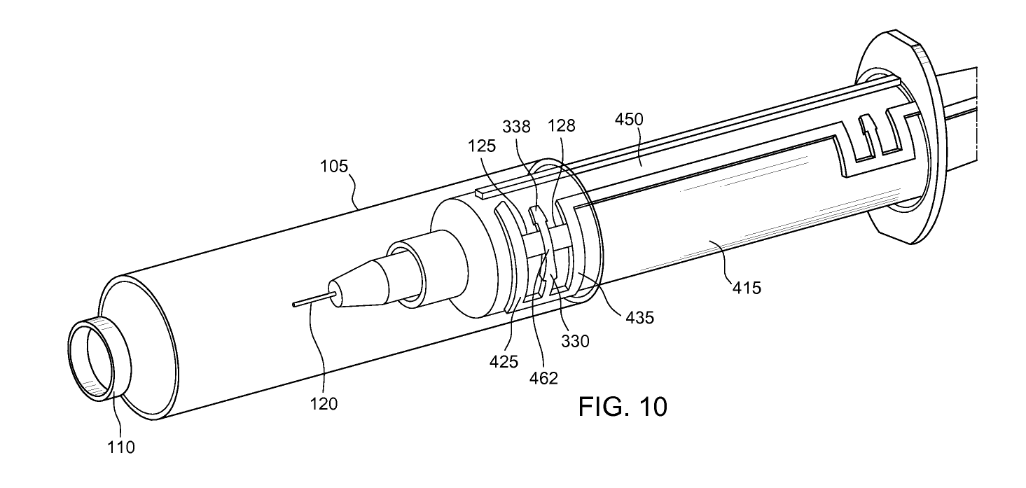

FIG. 9 depicts a perspective view of the sleeve 105 and the barrel 415 of FIG. 8, between an operating position and a safe position, according to embodiments of the invention. The first position is defined by the sleeve 105 being in a retracted position, wherein the needle 120 protrudes from the sleeve 105 (and/or is capable of traversing a channel 450 and is capable of being temporarily locked on a first locking tab or notch 422, i.e., an operating status). The second position is defined by the needle 120 being enclosed within the sleeve 105 (and/or is capable of being locked on and engaging a second locking notch 462). Referring now to facing the distal end 490 of the sleeve 105, the sleeve 105 is rotated counter-clockwise (or the barrel 415 is rotated clockwise) so that first and second protrusions 125, 128 are disengaged from the locking tab or notch 422. The sleeve 105 can slide toward the second position, with the first and second protrusions 125, 128 disposed within the channel 450 formed by guide tracks 410 and 412, as shown and then capable of being rotated again to engage the locking tab or notch 462.

FIG. 10 depicts a perspective view of the sleeve 105 and the barrel 415 in a temporarily locked second position, according to embodiments of the invention. For example, again referring to facing the distal end 490 of the sleeve 105, the sleeve 105 slides until the protrusions 125, 128 reach a position between the guide tracks 425 and 435. The sleeve 105 is rotated clockwise (or the barrel 415 is rotated counter-clockwise) so that the first and the second protrusions 125 and 128 travel past the tab or notch 338 and engaging the locking notch 462, which is the second temporarily locked position. The tab or notch 338 is a temporary locking position because the tab or notch 338 is not so wide as to prevent the counter-rotation of the sleeve 105, thereby allowing the protrusions 125, 128 to slide past and into the channel 450.

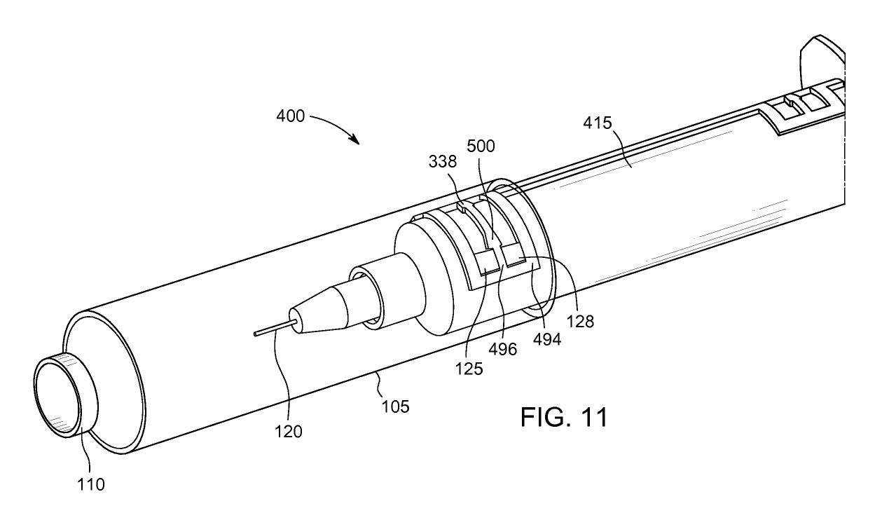

FIG. 11 depicts a perspective view of the sleeve 105 and the barrel 415 of FIG. 8 in a permanently locked safe position, according to embodiments of the invention. The sleeve 105 is rotated clockwise (or the barrel 415 is rotated counter-clockwise) so that the first and second protrusions 125 and 128 travel past the tab 500 and engage the locking notch 496, which is the third position, which permanently locks the barrel 415 and the sleeve 105. Because of the wide design of the tab 500, trying to rotate the sleeve 105 to the second unlocked position (described above) is not possible. Additionally, the sleeve 105 cannot be rotated further because of the stop 494. Accordingly, the needle 120 is permanently disposed within the sleeve 105 unless the safety syringe apparatus 400 is destroyed, rendering the safety syringe apparatus 400 incapable of re-use.

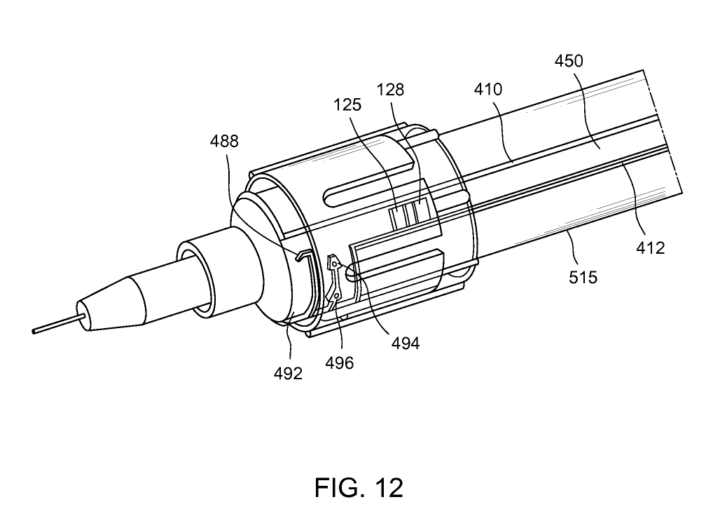

FIG. 12 depicts a perspective view of a collar 501 and a barrel 515 assembly, according to embodiments of the invention. FIG. 12 depicts the barrel 515 having a tapered tab 488 disposed on a proximal edge 492. Because the tapered tab 488 is tapered, the first and the second protrusions 125 and 128 can more easily be fitted into the channel 450. As shown, the barrel 515 comprises front temporary lock 494 and front permanent lock 496, which the first protrusion 125 and second protrusion 128 can engage, an operates similarly as described above with respect to locking features. The collar 501 fits on the barrel 515 and the collar 501 can also engage a cylinder, as discussed more fully below.

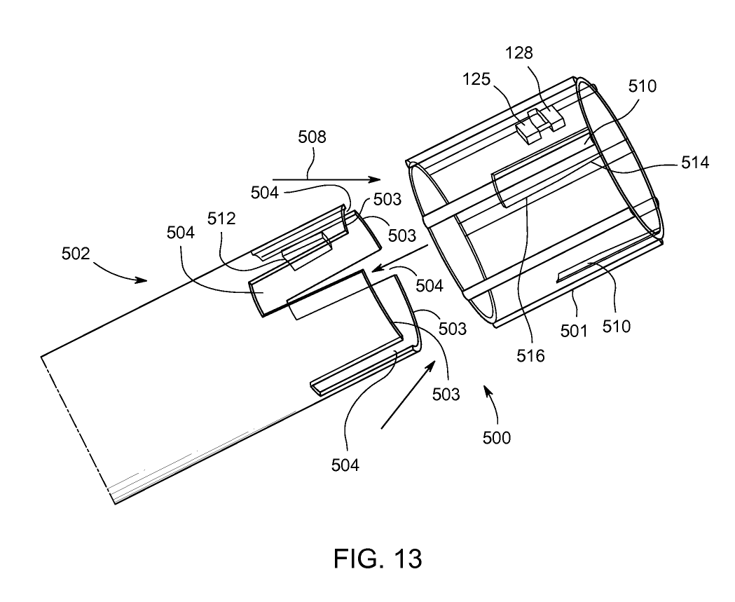

FIG. 13 depicts exploded perspective view of a two-piece sleeve 500 for mating with a barrel 515, according to embodiments of the invention. The two-piece sleeve 500 comprises a cylinder 502 and the collar 501, which can be forced together providing, for example, an interference fit. The two-piece sleeve 500 can be assembled with the barrel 515, as shown, or for example, the barrel 415 discussed above, by forcing the two-piece sleeve 500 and the barrel 515 together in direction 508. The barrel 515 is similar to the barrel 415 and comprises a temporary locking tab or notch 522 and a permanent locking tab or notch 524.

The cylinder 502 comprises petals 503 and are separated by windows 504. In some embodiments according to the invention, there are two, three, four, or more petals 503 and in some embodiments, the cylinder 502 has no petals 503 or windows 504. The petals 503 fold inward or otherwise fit into and travel inside of the collar 501 during assembly. In some embodiments, the collar 501 comprises a stiffer material than the sleeve 502 for ease of assembly and creating a tight, interference fit. The collar 501 comprises ramps 510, which mate with the slot 512 in the cylinder 502, so the two-piece sleeve 500 is releasably engageable. The ramps 510 optionally comprise draft angles of, for example, 1 to 5°, so that the ramps 510 are not as wide at a proximal end 514 compared with a distal end 516. The ramps 510 on the collar 501 engage with a releasable beam on a circumferential flange (as shown below) on the distal end of the barrel 415, which deters a user from disassembling the syringe apparatus. The collar 501 also comprises a first protrusion 125 and a second protrusion 128, as described above, for locking the two-piece sleeve 500 onto temporary or permanent locking notches also described above. Because the two-piece sleeve 500 can be separated, i.e., the cylinder 502 can be removed from the collar 501, during a procedure, the cylinder 502 can be removed and discarded without removing the collar 501 from the barrel 515, as described above.

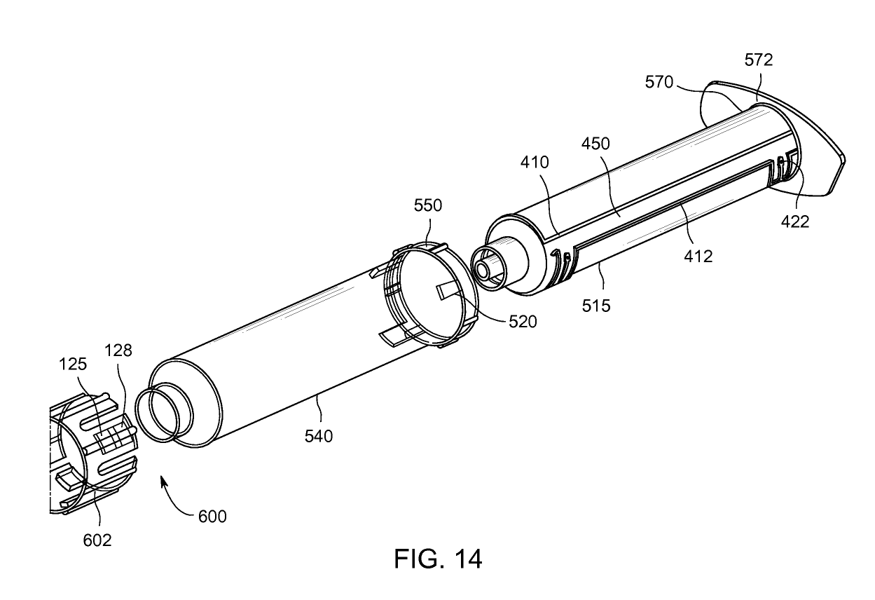

FIG. 14 depicts an exploded perspective view of a two-piece sleeve 600 for mating with the barrel 515, according to embodiments of the invention. As shown, the two-piece sleeve 600 comprises a cylinder 540 having a flange 550 and a collar 602. The collar 602 has first and second protrusions, such as the first and the second protrusions 125 and 128, disposed on an internal surface of the collar 602. The collar 602 fits on the outside of a cylinder 540, the first and the second protrusions 125, 128 mating with a window 504 on or near the flange 550. The two-piece sleeve 600 can be assembled with a barrel, for example, the barrel 515 discussed above, by forcing the two-piece sleeve 600 and the barrel 515 together. The first and the second protrusions 125, 128 travel into a channel 450 formed by the guide tracks 410, 412, where they can engage the temporary tab or notch 422 or another temporary or permanent lock, as discussed below. A releasable beam 570 on a circumferential flange 572 of the barrel 415 engages at least one ramp 510 to deter medical personnel from disassembling the syringe.

The exemplary embodiments were chosen and described to best explain the principles of the present disclosure, and its practical applications, to enable others skilled in the art to best utilize the invention and various embodiments with various modifications as may be suited to any particular use contemplated. Further embodiments of the invention may be devised without departing from the basic scope of disclosure herein, and the scope thereof is to be determined by the claims that follow. It is to be understood that any of the features of the safety syringe apparati, e.g., tabs, tracks, barrels, sleeves, protrusions, may be incorporated within any of the embodiments described herein without further recitation.

What is claimed is:

1. A safety syringe, comprising:
a needle;
a barrel having at least one locking track and at least one tab disposed on an exterior surface, wherein the barrel is in fluid communication with the needle;
a plunger capable of fitting into the barrel; and
a hollow sleeve having a first opening on a first end and a second opening on an opposite end, and having at least one protrusion on an interior surface of the hollow sleeve,
wherein the tab on the at least one locking track on the barrel is adapted to engage the at least one protrusion of the hollow sleeve, thereby locking a position of the barrel with respect to a position of the hollow sleeve.

2. The safety syringe of claim 1, wherein the barrel comprises at least two locking tracks, having at least one tab on each of the at least two locking tracks.

3. The safety syringe of claim 1, wherein the at least one locking track is disposed between two guiding tracks.

4. The safety syringe of claim 1, wherein a locking track comprises a tab for a temporary lock for use in a first position.

5. The safety syringe of claim 1, wherein the hollow sleeve obscures the needle in the safe position.

6. The safety syringe of claim 2, wherein the tab on at least one locking track comprises a temporary locking tab and at least one tab on at least one locking track comprises a permanent locking tab.

7. The safety syringe of claim 2, wherein at least one of the two locking tracks comprises at least two tabs.

8. The safety syringe of claim 7, wherein at least one of the at least two tabs comprises a temporary locking tab and at least one of the at least two tabs comprises a permanent locking tab.

9. The safety syringe of claim 8, wherein the temporary locking tab is positioned on a proximal portion of the track for locking the safety syringe in an operating position and the permanent locking tab is positioned on a distal portion of the track for locking the safety syringe in a safe position.

10. The safety syringe of claim 7, wherein the at least two tabs are tapered.

11. The safety syringe of claim 1, wherein a locking track comprises a tab for a temporary lock for use in a second position.

12. The safety syringe of claim 1, wherein a locking track comprises a tab for a temporary lock for use in a second position and a tab for a permanent lock for use in a third position.

13. A safety apparatus, comprising:
a collar; and
a cylinder adapted to mate with the collar to form a sleeve, wherein the collar comprises at least one protrusion on an interior surface of the collar and a first and a second opening;
wherein the sleeve is capable of engaging a barrel having at least one track having a tab, extending on a portion of an exterior circumference of the barrel and adapted to allow at least one protrusion of the collar to rotatingly engage the tab to lock a position of the barrel with respect to a position of the sleeve.

14. The safety apparatus of claim 13, wherein the barrel further comprises a releasable beam and a circumferential flange, and the collar comprises at least one ramp; wherein the releasable beam engages the at least one ramp, deterring medical personnel from disassembling the safety apparatus.

15. The safety apparatus of claim 13, wherein the at least one protrusion on the interior surface of the collar is adapted to engage the tab on the at least one track in an operating position.

16. The safety apparatus of claim 13, wherein the tab on the track is adapted for a temporary lock.

17. The safety apparatus of claim 13, wherein the tab on the track is adapted for a permanent lock.

18. A method for protecting a user from a needle stick, comprising:
aligning a barrel of a syringe having a track, that has a tab disposed on an exterior surface of the barrel, with a hollow sleeve having at least one protrusion disposed on an interior surface of the hollow sleeve; and
rotating at least one of the hollow sleeve or the barrel while a needle of the syringe is in an obscured position within the hollow sleeve,
wherein rotating the hollow sleeve engages the tab with the at least one protrusion on the barrel, thereby locking the position of the hollow sleeve with respect to the barrel, preventing a needle stick.

19. The method of claim 18, wherein the locking track comprises at least one of a temporary lock or a permanent lock.

20. The method of claim 18, wherein the locking track is on a portion of the barrel corresponding with a safe position.
